(19) United States
(12) Patent Application Publication
(10) Pub. No.: US 2020/0069474 A1
(43) Pub. Date: Mar. 5, 2020

(54) POSTOPERATIVE AUXILIARY APPARATUS

(71) Applicant: Waldir REN, Guaratinguetá (BR)

(72) Inventor: Waldir REN, Guaratinguetá (BR)

(21) Appl. No.: 16/613,415

(22) PCT Filed: Jun. 12, 2018
(86) PCT No.: PCT/BR2018/050188

§ 371 Date: Nov. 14, 2019

(30) Foreign Application Priority Data
Jun. 13, 2017 (BR) 10 2017 012569.6
May 17, 2018 (BR) 10 2018 010059-9

(51) Int. Cl. A61F 13/00

(52) U.S. Cl. CPC A61F 13/00017

(57) ABSTRACT

A postoperative auxiliary apparatus, comprising an apparatus built of low-density polyethylene, coated with elastic cotton fabric, with a shape capable of transmitting a resulting pressure, which is concentrated by supporting means by bandage, elastic fabrics or cotton fabrics, and said pressure assists both in healing burns and in decreasing keloids, in supporting body parts, accelerating these procedures compared with other techniques.

The present application for patent of invention claims national priority of proceeding BR 10 2017 012569-6, filed on Jun. 13, 2017, under the terms of Law No. 9,279 of May 14, 1996.

### FIELD OF INVENTION

The present invention relates to the field of application of medical apparatuses, more specifically the field of application of auxiliary apparatuses for use in postoperative treatments.

The purpose of the invention is to present a medical apparatus to assist with postoperative treatments enabling the effects of compression, containment and expansion, and can thus being used in the treatment of keloid, absorption of edema and swelling or even in the modeling and expansion of certain body areas.

### BACKGROUND OF THE INVENTION

Brazil is the second country in plastic surgery numbers and aesthetic procedures in the world, behind only the USA. Studies have shown that in the year 2015, 1.2 million plastic surgeries and 1.1 million aesthetic procedures were performed in Brazil, and 1.4 million of surgical procedures were performed in the USA. This being the case, much has progressed with regard to these types of procedures both in the procedures per se and in postoperative treatment.

A demand that has grown much with the occurrence of these types of procedures concerns postoperative treatment, since this type of procedure requires a follow-up, in certain cases, which is why it is referred to in this step, since the expected result of such procedures is an improvement or aesthetic correction in the patient.

By their own characteristic, aesthetic procedures tend mostly to be invasive for patients and often require a long time in surgical techniques, in many cases exceeding 2 hours of procedures. Another characteristic of this type of procedure is the symmetry required in some specialties, such as facial surgeries, where in the first moment one side of the patient's face is worked on and after hours of surgery the other side is worked, demanding not only greater surgical care by the surgeon, but also patient care in order to maintain the side that was first operated free of alterations and problems.

In most cases of surgical procedure of an aesthetic nature both in the procedures itself and in the postoperative period, surgeons use elastic bandages to restrict the operated site and thus ensure the results obtained. However the pressure exerted by this type of bandage on the site itself is far from ideal and highly related to the pressure exerted at the time of the bandaging, that is, if the surgeon pulls the bandage tightly, it may result in some kind of deformation at the site and contribute negatively to the desired result.

Accordingly, some surgeons chose to use banding and modeling braces, which are marketed for each type of surgery, with abdominal and pectoral bands and so on. Such bands overcome the question of pressure tightness, since they have standard sizes for their use: small (S), Medium (M) and Large (L). However, since such bands and braces cover regions entirely, they do not allow the concentration of pressure in the most critical region, for example, an abdominal keloid, which makes it difficult to obtain positive results or increases the time until this result is obtained.

So some surgeons chose to use containment plates in postoperative treatment, these plates being reduced in size and in front of the post-surgical belts and bands, contributing to a more localized tension. However, such plates still depend on the straps, bands or even bandages to remain in the proper position, that is, the use thereof is still subject to the same drawbacks and disadvantages arising from the use of these accessories. Another disadvantage in using this type of plate lies in the fact that the material used in its manufacture is rigid, which in many cases ends up creating discomfort for patients and in certain uses even hurting and injuring them.

Therefore, it is noted that the state of the art is lacking in a solution that simultaneously provides comfort for the patient, localized tensioning, ease of application and independent of medical expertise at the time of application.

### BRIEF DESCRIPTION OF THE INVENTION

The purpose of the present invention is to present an apparatus for postoperative treatment of various surgeries, especially surgeries and aesthetic procedures, which allows easy application of a localized tension and does not create discomfort for patients.

The objective of said auxiliary apparatus is achieved due to its construction in low density polyethylene, which allows the absorption of part of the applied tension thereon by the cotton band or bandage, thus allowing the tension exerted on the site of interest not to be excessive.

Another objective of said invention lies in the fact that the treatment with the auxiliary apparatus considerably reduces postoperative treatment time and also increases the level of natural healing in cases of burns.

### BRIEF DESCRIPTION OF THE DRAWINGS

The subject matter that is the object of this invention will be entirely explained in terms of technical aspects following the detailed description made based on the drawings listed below, wherein:

FIG. 1 presents a top view of the apparatus in its support application;

FIG. 2 presents a front view of the apparatus in its support application;

FIG. 3 presents a perspective view of the apparatus in its support application;

FIG. 4 presents a front view of the apparatus in its expansion application;

FIG. 5 presents a rear view of the apparatus in its expansion application;

FIG. 6 presents a side view of the apparatus in its support application;

FIG. 7 presents a perspective view of the apparatus in its compression application;

FIG. 8 presents a side view of the apparatus in its compression application;

FIG. 9 presents a front view of the apparatus in its compression application;

FIG. 10 presents a top view of the apparatus in its compression application;

FIG. 11 presents a perspective view of the apparatus in its modeling application;

FIG. 12 presents a side view of the apparatus in its modeling application;

FIG. 13 presents a front view of the apparatus in its modeling application; and

FIG. 14 presents a top view of the apparatus in its modeling application.

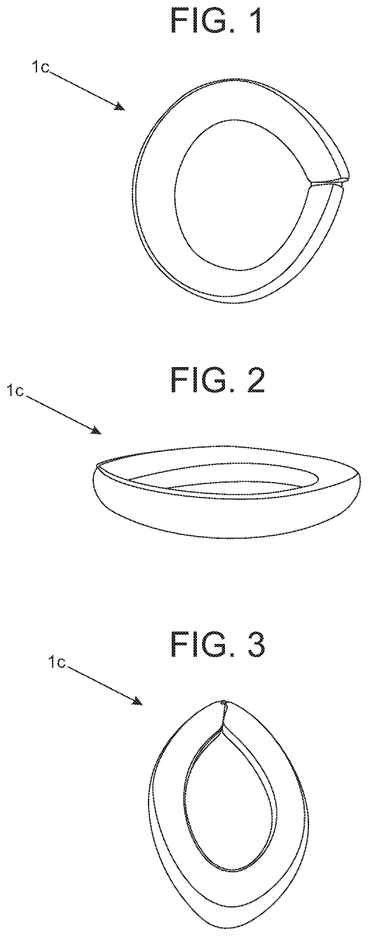

### DETAILED DESCRIPTION OF THE INVENTION

In accordance with the objectives presented by way of the brief description, the present patent application “POSTOPERATIVE AUXILIARY APPARATUS” presents an apparatus (1) built of low-density polyethylene and coated with elastic cotton fabric, where the apparatus (1) has a form capable of transmitting a pressure resulting from the means whereby such apparatus (1) is sustained and this may be bandages or elastic cotton fabrics.

The main and inherent characteristic of said apparatus (1) is that it is made of low-density polyethylene and provides a certain degree of deformation in its structure, and this deformation is capable of decreasing the pressure exerted by supporting the apparatus (1) such that it provides localized pressure, which continues through its extension in contact with the patient, but not excessive, thus avoiding the feeling of discomfort for the patient due to the use thereof.

The apparatus (1) is also characterized by having a coating made of elastic cotton fabric, which makes it easy to clean, since the apparatus (1) will always be in contact with body parts subject to various discharges such as blood, pus and the like as well as medicines for better healing of the application region. In this way, the coating on the elastic cotton fabric will allow the apparatus (1) to be washed and subsequently reused.

Another important characteristic of the apparatus (1) and which is also inherent in its construction of low-density polyethylene lies in its impermeability which contributes both to cleanliness and sanitization, as well as the correct administration of the medicines at the site of contact with the patient's skin, since the apparatus (1) will not uptake part of this medicine.

The apparatus (1) basically has three main uses, one being an application for the effect of compression, one application for the effect of contention and modeling and one application for the effect of expansion.

The apparatus (1) in its application for the effect of compression is used mostly for control and decrease in keloids, which mainly focus on incisions or cuts resulting from aesthetic procedures, as well as in the healing of burns or decreased scars resulting from aesthetic procedures.

Said apparatus (1) in its application for the effect of compression has its faces slightly domed, with positive curvature, that is, outward, so that the apparatus (1) allows for localized compression in the patient's body, stimulating the blood flow in the region without overly shaping the region, thus increasing the speed of healing.

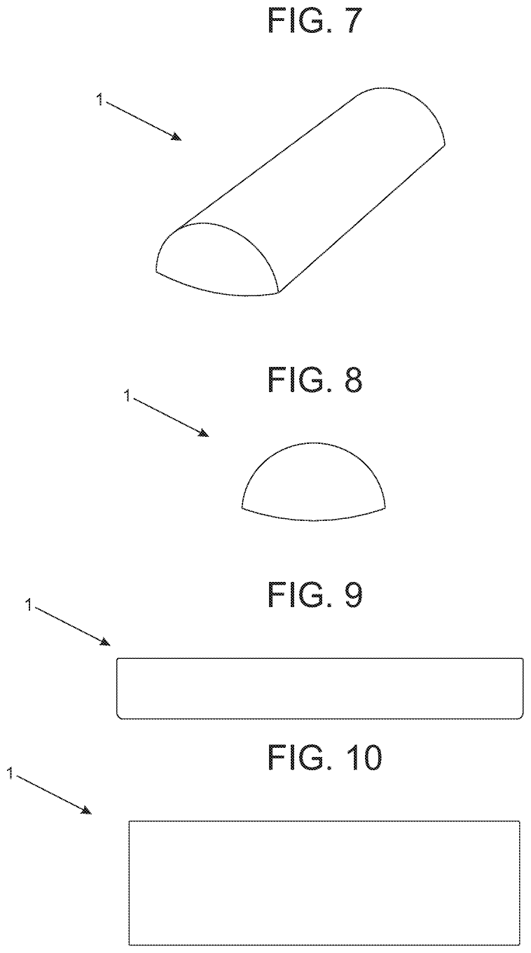

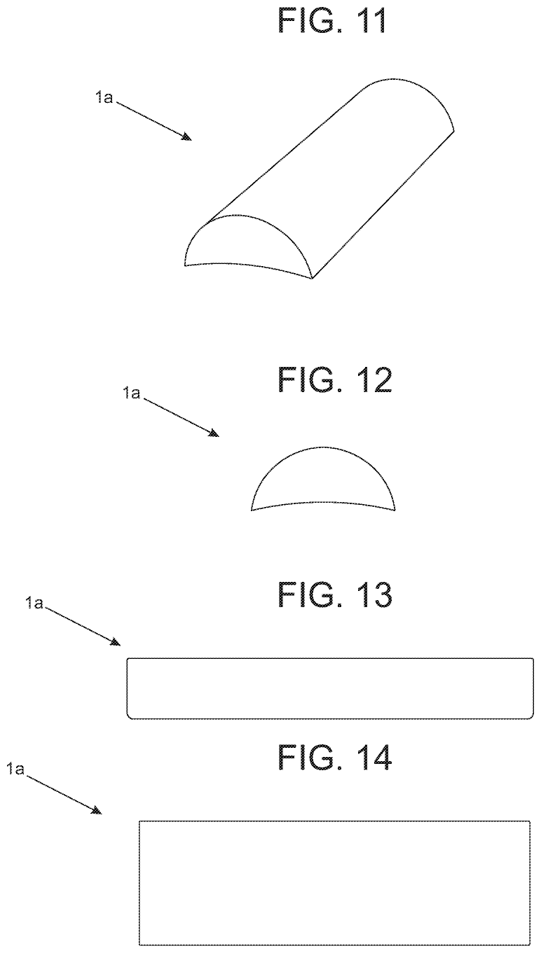

FIG. 7 presents a comparison between a scar in its initial state and the evolution of the same scar after the use of an apparatus (1) in its application for the effect of compression for two and for three days, where it is easy to identify the reduction of both length of the scar and its height in relation to the other regions of the body.

The apparatus (1) in its application for the effect of compression is mostly used to aid the absorption of edema and swelling resulting from aesthetic procedures, where the accumulation of liquids in localized regions of the patient's body is extremely common. Accordingly, the apparatus (1) has one of its straight faces opposite the other slightly positively domed face, that is, with outward curvature. Such constructivity of the apparatus (1) favors localized compression of these edema and swelling so that the accumulated fluids are gradually scattered in the regions near the initial swelling and consequently absorbed by the body due to increased blood circulation in the region. The presence of a straight face in the apparatus (1) also prevents too much compression of the region and consequent deformation thereof since it will compress the swelling or edema up to the height of the skin nearby, favoring a more homogeneous result of the procedure.

In its application for modeling, the apparatus (1a) presents one of its faces slightly negatively domed, that is, having inward curvature. Such application is mostly used in postoperative treatments in aesthetic procedures where the final result requires stabilization such as facelift surgeries. In these cases, the apparatus (1a) allows the blood flow of the region to be stimulated by a slight pressure so that the body adapts more easily to the changes performed in the surgery.

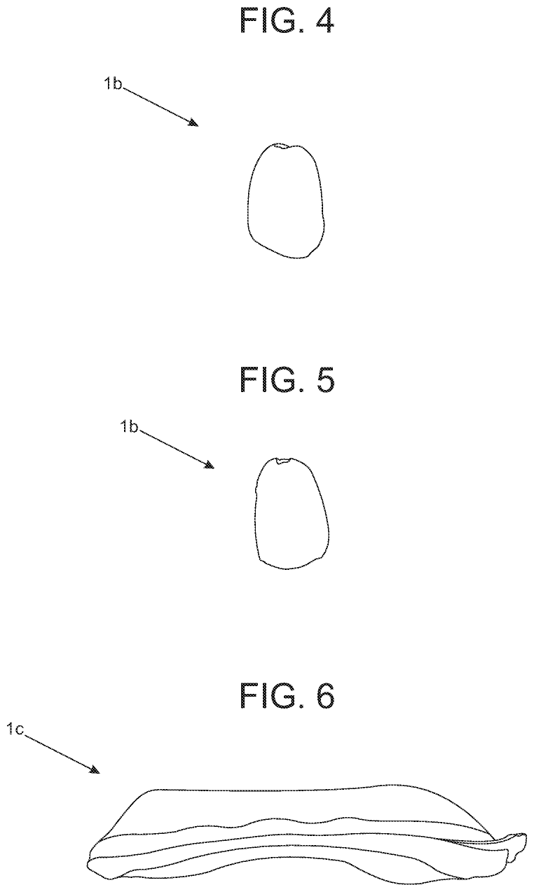

The apparatus (1b), in its application for the effect of expansion is mostly used in post-surgical treatments that require reconstruction or remodeling of rounded areas such as surgeries in the abdominal region requiring reconstruction of the navel. Accordingly, the apparatus (1b) has an essentially truncated cone shape, with two ends having different diameters, a smaller diameter and a larger diameter, where the larger diameter end has a straight face and the smaller diameter has a positively domed end, that is, with outward curvature, as shown in FIG. 4. This format allows the supporting means of the apparatus (1b) to exert pressure on the larger face so that the smaller face—in contact with the patient's body—exerts a great enough pressure to the point of allowing the apparatus (1b) to enter into the skin region slightly, creating a hole.

Said invention further has a support application, where the apparatus (1c) consists of an apparatus (1a) having joined ends, forming a circular padding, this being ideal to support spherical body portions such as the head, buttocks, etc.

The major advantage of using the apparatus (1) with application for the effect of expansion lies in the fact that it does not require a cut in the patient's skin, the pressure exerted on the apparatus itself and consequent pressure exerted by the apparatus being the sole means of expanding the region and creating the hole, by way of the smaller diameter, in the patient, without the need for invasive procedures.

By definition of application, the pressure exerted on the skin will be transmitted to the tissues below it, acting in the treatment of the accumulation of fluids dammed up in the subcutaneous tissue, especially after liposuction and surgical procedures such as skin grafts and displacement of cutaneous and muscle-cutaneous patches. Due to the high density of the skin (1.05 g/cm³), the energy emitted on its surface by the apparatus (1) is dissipated and becomes a roller compressor over the subcutaneous area where the density is lesser (0.9 g/C³).

By this compression mechanism, excess fluids (edema and blood fluids) will migrate to the periphery of the cushion looking for areas of low resistance away from scars and anatomical points of skin adherence to deep planes, where this excess will be absorbed and drained by the lymphatic and venous vessels. This apparatus (1) also has application in areas of chronic retention of liquids in the dermis and below this (cellulitis, lymphatic stasis), and in protectors, immobilizers and body supports.

As for application of the apparatus (1) on hypertrophic scars or even on keloid scars on burns, current compressive garments, despite exerting strong and broad pressure (between 40 and 90 mmHg), are not sufficient for an efficient treatment. By associating the apparatus (1) and (1a) in the selected areas, the pressure exerted thereby allows a reduction in the pressure of the compressive garments in the other body areas, making it more comfortable and stimulating the use of these garments which, per se, cause suffering and stress to the patient.

To be effective and safe, the apparatus (1.1a, 1b and 1c) must have a density in the interface between water and fat. In this sense, the appropriate material should be soft, low resilience, waterproof, flexible, light, inert and nontoxic, low density polyethylene being ideal, because it has a density between 0.910 and 0.925, allowing skin compression without obstructing the blood and lymphatic vessels on and under the skin.

It is known that in the human body the pressure in the terminal vessels is 8.0 mmHg. As the density of the apparatus (1) is similar to that of the subcutaneous tissue, the pressure exerted on the subcutaneous tissue does not exceed the pressure of the terminal vessels, thus avoiding ischemia and consequent necrosis of the skin and subcutaneous area.

The effective tension variation of the compression vest on the apparatus (1.1a, 1b and 1c) is related to the state of the skin. Normal skin requires a pressure between 10 and 40 mmHg while healed and stiffened skin offers a higher resistance, between 40 and 90 mmHg.

The advantages present in the apparatus (1) over the state of the art are diverse, and particularly include the fact that the apparatus (1) transfers a localized pressure while the options presented in the state of the art exert pressure in areas and the fact that the apparatus (1) is easy to apply on patients and does not proliferate a feeling of discomfort.

Another advantage present in the apparatus (1) which overcomes the options presented in the state of the art lies in the fact that it is waterproof, facilitating cleaning and sanitization in terms of maintenance of the medicines applied jointly thereby in the region of application on the patient.

Lastly, another advantage present in the apparatus (1) is not found in the state of the art refers to its use, which allows three types of effects to be obtained, namely an application with the effect of compression, an application with the effect of modeling and contention and, further, an application with the effect of expansion.

It should be understood that the present description does not limit the application to the details described herein and that the invention is capable of other modalities and can be carried out or executed in a variety of ways, within the scope of the claims. Although specific terms have been used, such terms should be interpreted in a generic and descriptive sense, and not with the purpose of limitation.

What is claimed is:

1. A “POSTOPERATIVE AUXILIARY APPARATUS”, characterized in that it presents an apparatus (1), built of low-density polyethylene, coated with elastic cotton fabric, in which the apparatus (1) has a shape capable of transmitting a pressure resulting from the means whereby said apparatus (1) is supported, and can be bandages or elastic cotton fabrics, having a compression application, a support application, a modeling application and an expansion application.

2. The “POSTOPERATIVE AUXILIARY APPARATUS”, according to claim 1, characterized in that the apparatus (1) in its compression application, presents a shape with its faces slightly domed, with positive curvature, that is, outward, such that the apparatus (1) allows localized compression on the patient's body, stimulating the blood flow in the region without excessively shaping the region.

3. The “POSTOPERATIVE AUXILIARY APPARATUS”, according to claim 1, characterized in that the apparatus (1) in its modeling application, presents a shape containing on one of its faces slightly negatively domed, that is, with inward curvature.

4. The “POSTOPERATIVE AUXILIARY APPARATUS”, according to claim 1, characterized in that the apparatus (1) in its expansion application, has an essentially truncated cone shape, with two ends having different diameters, a smaller diameter and a larger diameter, where the end with the larger diameter has a straight face and the end with the smaller diameter has a positively domed end, that is, with outward curvature.

5. The “POSTOPERATIVE AUXILIARY APPARATUS”, according to claim 1, characterized in that the apparatus (1) in its support application has a shape with joined ends, forming a circular padding and one of its faces is slightly negatively domed.
